(19) United States
(12) Patent Application Publication
(10) Pub. No.: US 2012/0231473 A1
(43) Pub. Date: Sep. 13, 2012

(54) FLOW CYTOMETRY METHOD THROUGH THE CONTROL OF FLUORESCENCE INTENSITIES

(75) Inventor: Kyung Ja Han, Gyeonggi-do (KR)

(73) Assignee: CATHOLIC UNIVERSITY INDUSTRY ACADEMIC COOPERATION FOUNDATION, Seoul (KR)

(21) Appl. No.: 13/510,254

(22) PCT Filed: Nov. 24, 2010
(86) PCT No.: PCT/KR10/08366

§ 371 Date: May 16, 2012

(30) Foreign Application Priority Data
Nov. 24, 2009 (KR) 10-2009-0113899
Nov. 24, 2010 (KR) 10-2010-0117617

(51) Int. Cl. G01N 21/64; C12M 1/34

(52) U.S. Cl. 435/7.1; 435/287.2

(57) ABSTRACT

Provided is a flow cytometry method including adjusting cell populations targeted by antibodies conjugated with a same-color fluorochrome to show different fluorescence intensities according to types of the antibodies. Unlike a conventional flow cytometry method capable of classifying a positive and negative of one target using one antibody per color, the flow cytometry method adjusts several types of antibodies conjugated with a single-color fluorochrome to respectively show different fluorescence intensities, or adjusts the amounts of antibodies conjugated with a fluorochrome differently according to types of the antibodies, thereby classifying a positive and negative of multiple targets using one color. Accordingly, even when a current flow cytometer capable of classifying a limited number of colors is used, it is possible to classify a variety of cell populations to be clinically examined.

### CROSS-REFERENCE TO RELATED APPLICATION

This application claims priorities to and the benefit of Korean Patent Application No. 10-2009-0113899 filed on Nov. 24, 2009 and Korean Patent Application No. 10-2010-0117617 filed on Nov. 24, 2010, the disclosures of which are incorporated herein by reference in their entirety.

### BACKGROUND

1. Field of the Invention

The present invention relates to a flow cytometry method through the control of fluorescence intensities.

2. Discussion of Related Art

Although most hematologic analyzers are good at quantitative enumeration of leukocytes, a microscopic examination of blood smears is required to ascertain the presence of abnormal cells. The manual microscopic sorting of leukocytes is not suitable as a reference method due to a lack of qualitative parameters and inaccuracy. In such case, immunophenotypic characterization of abnormal cells by flow cytometry is necessary to readily examine a cytological abnormality and obtain a final diagnostic conclusion. One of theoretical advantages of immunological recognition by cells based on flow cytometry would be identifying cell types beyond the possibility of cytology using various parameters.

Recent research has reported that a method of classifying leukocytes including abnormal cells by 5-color flow cytometry is reliable and may be used to classify 11 different types of cells. Thus far, such a method may be used to detect 20 different cell subsets including normal peripheral blood, and lymphocyte subsets, granulocyte and monocyte subsets, dendritic cell subpopulations, circulating progenitor and immature cells and other rare cells. However, one color can be used for only one antibody, and positive and negative cell populations can only be shown per color. Also, there is the limited number of colors that can be classified by a flow cytometer, and thus different cell populations are not sufficiently indentified. For this reason, any of commercially available reagents for flow cytometry of a 5- or 6-color sample may not be used as a single combination of markers to count all the cell populations. To overcome the limited number of antibodies that can be mixed in one tube, sequential gating has been attempted. However, a very complex gating strategy should be used to classify up to 11 different types of cells. Meanwhile, a one-tube immunophenotyping panel for classification of leukocytes and platelets has been reported. Faucher et al. have reported a single 6-antibody/5-color combination in a single tube method for counting five circulating blood cells in addition to undifferentiated cells. However, these methods can merely classify positive and negative cell populations per fluorochrome.

Flow cytometers currently used in most clinical laboratories can classify five colors. Thus, most flow cytometers provide at least seven parameters including a forward scatter (FSC), a side scatter (SSC), and five types of monoclonal anitbodies for labeling respective cells with different fluorochromes. Typical leukocyte populations can be classified using a cluster of differentiation (CD)-45 expression pattern. Since a CD45 expression pattern has been usually used for gating, only one color must be used to analyze CD45. Since cells show only positive and negative results, only 16 (2⁴) types of cell subpopulations (targets) can be classified by a 5-color flow cytometer.

However, the use of a single tube having five antibodies is insufficient to detect and characterize hematologic malignant cells in a single tube having five antibodies. More antibodies are necessary to classify more cell populations, and at least 20 types of antibodies are needed to analyze hematologic malignancy. More colors may be used to examine more types of cell populations, but an increased number of test tubes results in an increase of labor cost relating to flow cytometry, sample preparation/acquirement time, and post-acquirement analysis.

### SUMMARY OF THE INVENTION

The present invention is directed to providing a new flow cytometry method whereby a plurality of targets can be classified using a single color.

One aspect of the present invention provides a flow cytometry method including adjusting cell populations targeted by fluorochrome-conjugated antibodies to show different fluorescence intensities according to types of the antibodies.

The present inventors have designed the method based on the point that several targets should be classified even when using one fluorochrome in order to make an accurate hematologic diagnosis using a current flow cytometer which can classify a limited number of colors.

When many cell populations rather than two or three types can be classified using two or three types of monoclonal antibodies conjugated with one fluorochrome, the number of classifiable cell populations may increase by geometric progression.

Unlike a conventional cytometry method used to target one cell population using one antibody per color, the flow cytometry method of the present invention uses several types of antibodies conjugated with a single-color fluorochrome, and thus adjusts cell populations targeted by the several types of antibodies to show different fluorescence intensities according to types of the antibodies.

Adjusting the cell populations to show different fluorescence intensities may include adjusting fluorescence intensities of cell populations targeted by the antibodies in different levels by adjusting the several types of antibodies conjugated with the single-color fluorochrome to respectively show different fluorescence intensities or adjusting the amounts of the antibodies conjugated with the fluorochrome differently according to the types of the antibodies, but the present invention is not limited thereto.

As can be seen from embodiments to be described below, flow cytometry was performed by adjusting two types of monoclonal antibodies labeled with the same fluorochrome to show different fluorescence intensities. As a result of the flow cytometry, three kinds of cell populations were clearly classified, and thus the method of the present invention showed excellent repeatability. The coefficients of variation (CV) of lymphocyte subpopulations were similar to general flow cytometry data, and single-color 3-target flow cytometry showed similar results for all the cell populations, compared to general multicolor flow cytometry. Likewise, when flow cytometry was performed by adjusting three types of monoclonal antibodies labeled with the same fluorochrome to show different fluorescence intensities, four kinds of cell populations were clearly classified.

As such, assuming that three or four kinds of leukocyte cell populations can be classified using two or three kinds of monoclonal antibodies conjugated with a fluorochrome, when a flow cytometer which can classify five colors is used, 81 (3) kinds of cell subpopulations or 256 (4⁴) kinds of leukocyte cell subpopulations can be theoretically classified because one of the five colors is used for CD-45/side scatter (SSC) gate which classifies leukocytes.

However, the method of the present invention can be achieved even without using antibodies conjugated with a fluorochrome whose fluorescence intensity is differently adjusted in several levels. Adjusting the different cell populations to show different fluorescence intensities may include adjusting the amounts of the antibodies conjugated with the fluorochrome in different levels according to the types of the antibodies. Surprisingly, it was confirmed from embodiments described below that the method of the present invention can be implemented using antibodies conjugated with fluorochromes having the same fluorescence intensities without adjusting the intensity of the fluorochromes conjugated with the respective antibodies by adjusting amounts of a non-conjugated antibody and an antibody conjugated with a fluorochrome in different levels according to types of the antibodies.

Apart from the fact that several types of antibodies conjugated with a single-color fluorochrome are adjusted to respectively show different fluorescence intensities, or the amounts of antibodies conjugated with a fluorochrome are adjusted in different levels according to types of the antibodies, the flow cytometry method of the present invention may use a known flow cytometry method.

For example, the flow cytometry method may include: incubating an antibody composition in which the several types of antibodies conjugated with the single-color fluorochrome are adjusted to respectively show different fluorescence intensities or an antibody composition in which the amounts of the antibodies conjugated with the fluorochrome are adjusted in different levels according to the types of the antibodies together with a specimen; and gating the incubated specimen on a flow cytometer.

In one embodiment, adjusting the several types of antibodies conjugated with the single-color fluorochrome to respectively show different fluorescence intensities or adjusting the amounts of antibodies conjugated with the fluorochrome differently according to the types of the antibodies may be performed using fluorochromes with single or multiple colors. For example, as in Examples 1 and 2, a fluorochrome with one color may be adjusted to have several fluorescence intensities, and fluorochromes with the other colors conjugated to one antibody may be used as they are. Alternatively, as described in Example 3, a plurality of fluorochromes may be adjusted to have several fluorescence intensities, and the other fluorochromes conjugated to one antibody may be used as they are. Those of ordinary skill in the art may select any of antibodies whose fluorescence intensities will be adjusted for use.

When several types of antibodies conjugated with a single-color fluorochrome are adjusted to show different fluorescence intensities, it is ideal to adjust the intensity of the single-color fluorochrome conjugated to the antibodies in several levels in order to label cell populations, which have been targeted by the several types of antibodies, with one color.

In embodiments described below, since it was difficult to form a fluorochrome showing several intensities in a laboratory, antibodies conjugated with a single-color fluorochrome were used at a lower than normal concentration, or antibodies whose fluorescence intensities are reduced due to due to expiration of the period of validity were used. However, in preferred embodiments of the present invention, adjusting the several types of antibodies conjugated with the single-color fluorochrome to respectively show different fluorescence intensities may be performed by forming the single-color fluorochrome conjugated to the antibodies to have different fluorescence intensities. All commercially available fluorochrome-conjugated antibodies that are currently supplied by the same manufacturer have the same fluorescence intensity. However, when antibodies conjugated with a fluorochrome whose fluorescence intensity is adjusted in several different levels can be produced for use, it will be more desirable to implement the method of the present invention.

However, as described above, it is possible to target different cell populations by adjusting the amounts of antibodies conjugated with a fluorochrome differently according to types of the antibodies without adjusting an intensity of the fluorochrome conjugated to the antibodies. Thus, the commercially available fluorochrome-conjugated antibodies used in the art may be used in themselves.

When the different types of cell populations are adjusted to respectively show different fluorescence intensities, the difference between the fluorescence intensities should be within such a degree that can be detected by a flow cytometer. In one embodiment, the different fluorescence intensities are adjusted so that the fluorescence intensities of the different types of cell populations can differ from each other by 2 to 50 times, but the present invention is not limited thereto.

Any known fluorochromes may be used as the fluorochrome used in the flow cytometry method of the present invention. For example, the fluorochrome may be selected from the group consisting of fluorescein isothiocyanate (FITC), Alexa Fluor 488, green fluorescent protein (GFP), carboxyfluorescein succinimidyl ester (CFSE), carboxyfluorescein diacetate succinimidyl ester (CFDA-SE), DyLight 488, phycoerythrin (PE), propidium iodide (PI), peridinin chlorophyll protein complex (PerCP), PerCP-Cy5.5, PE-Alexa Fluor 700, PE-Cy5 (TRI-COLOR), PE-Cy5.5, PE-Alexa Fluor 750, PE-Cy7, allophycocyanin (APC), APC-Cy7, APC-eFluor 780, Alexa Fluor 700, Cy5, Draq-5, Pacific Orange, Amine Aqua, Pacific Blue, 4′,6-diamidino-2-phenylindole HCl (DAPI), Alexa Fluor 405, eFluor 450, eFluor 605 Nanocrystals, eFluor 625 Nanocrystals, and eFluor 650 Nanocrystals.

Also, any antibodies against an antigen to be analyzed by flow cytometry can be used in the flow cytometry method of the present invention. For example, the antibodies may be directed against an antigen selected from the group consisting of CD3, CD4, CD5, CD8, CD19, CD45, and CD56, but the present invention is not limited thereto.

The flow cytometry method of the present invention may be applied to all known flow cytometers. In one embodiment, flow cytometry may be performed by a flow cytometer equipped with a detector capable of classifying three to eight colors. In general, currently used flow cytometers have a detector capable of classifying five colors, but a flow cytometer equipped with a detector capable of classifying seven colors as described herein is also under development. As various colors are classifiable by a flow cytometer, the number of targets detectable by the present invention increases, so that the number of classifiable cell populations can increase by geometric progression.

Another aspect of the present invention provides a method of producing an antibody for flow cytometry, the method including conjugating respective single-color fluorochromes showing different fluorescence intensities with different types of antibodies, respectively. The antibody produced in this method may be usefully used for a flow cytometry method according to the present invention.

Still another aspect of the present invention provides an antibody composition including an antibody conjugated with a fluorochrome and a non-conjugated antibody. Here, an amount of the antibody conjugated with the fluorochrome is adjusted so that fluorescence intensities of cell populations can be adjusted in different levels in flow cytometry. Using an antibody conjugated with a conventionally used general fluorochrome, a user may be able to produce and use an antibody composition to have different fluorescence intensities according to cell populations, but it is difficult to individually produce and use the antibody in a clinical scene. Thus, when the above-mentioned antibody composition is provided, flow cytometry will be performed more rapidly and conveniently.

Still another aspect of the present invention provides a computer-readable recording medium storing a program for analyzing distribution of cells targeted by different antibodies in a specimen, the program executing in a computer system the steps of: recognizing different fluorescence intensities of cells targeted by different antibodies; and classifying the cells according to the different fluorescence intensities to analyze distribution of the cells targeted by the different antibodies in a specimen.

Yet another aspect of the present invention provides a flow cytometer including the computer-readable recording medium.

As described above, the flow cytometry method according to the present invention may use a conventional flow cytometer as it is, but a modified flow cytometry program suitable for the flow cytometry method of the present invention is more preferred.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows representative dot plots and histograms of single-color 3-target flow cytometry for two analysis samples;

FIG. 2 shows representative dot plots and a histogram of single-color 4-target flow cytometry; and

FIG. 3 shows representative dot plots of 2-color 9-target flow cytometry for two analysis samples, in which FIGS. 3A and 3B show results obtained using a bone marrow biopsy material of a mantle cell lymphoma patient, and FIG. 3C shows results obtained using peripheral blood of a normal individual.

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

### EXAMPLES

Flow cytometry was performed as described in Examples 1 to 3 to determine whether different cell populations could be classified using a method of adjusting several types of antibodies conjugated with a single-color fluorochrome to respectively show different fluorescence intensities.

Before flow cytometry was performed, analytic samples and antibodies were prepared. 20 peripheral blood samples that were allotted to a laboratory after general blood cell counting and had a normal number of cells were used for flow cytometry. The peripheral blood samples were kept in an empty plastic tube having an inner wall coated with K2-EDTA (Becton Dickinson, Franklin lakes, N.J., USA), and were used for flow cytometry within four hours after blood sampling. One bone marrow biopsy sample associated with mantle cell lymphoma was also used as the analysis sample.

The antibodies used for flow cytometry were purchased from Beckton Dickinson immunocytometry systems (San Jose, Calif., USA), including an antibody against CD3, CD4, CD5, and CD19 conjugated with fluorescein isothiocyanate (FITC), an antibody against CD19, CD4, and CD56 conjugated with phycoerythrin (PE), and an abtibody against CD45 conjugated with peridinin chlorophyll protein complex (PerCP). In order to label several types of antibodies with one color, it is ideal to adjust the intensity of a single-color fluorochrome conjugated to the antibodies in several levels. However, since it is difficult to form a fluorochrome showing several intensities in a laboratory, antibodies conjugated with a single-color fluorochrome were used at a lower than normal concentration, or antibodies whose fluorescence intensities had been reduced due to expiration of the period of validity was used. Monoclonal antibody cocktails used in Examples 1 to 3 are as shown in Table 1 below.

TABLE 1
Antibody Cocktail Used in Single-Color Multi-Target Flow Cytometry

| | | Fluorochrome (volume/test) | | | | | |
|---|---|---|---|---|---|---|---|
| | Antibody Cocktail | FITC | | PE | | | PerCP |
| Example 1 | CD3 + CD4/—/CD45 | CD3 | CD4 | | | | CD45 |
| | | (0.1 uL) | (5 uL) | | | | (5 uL) |
| | CD19 + CD3/—/CD45 | CD19 | CD3 | | | | CD45 |
| | | (0.5 uL) | (5 uL) | | | | (5 uL) |
| Example 2 | —/CD56 + CD19 + CD4/CD45/— | | | CD56 | CD19 | CD4 | CD45 |
| | | | | (0.1 uL) | (0.5 uL) | (20 uL) | (5 uL) |
| Example 3 | CD5 + CD3/CD19 + CD4/CD45 | CD5 | CD3 | CD19 | CD4 | | CD45 |
| | | (0.1 uL) | (5 uL) | (0.5 uL) | (5 uL) | | (5 uL) |
| | CD3 + CD4/CD25 + CD19/— | CD3* | CD5 | CD25* | CD19 | | |
| | | (5 uL) | (5 uL) | (5 uL) | (5 uL) | | |

*Period of Validity: CD3: Jul. 01, 2004, CD25: Jun. 30, 2004

As a flow cytometer, a 7-color BD FACSCanto II flow cytometer (Becton Dickinson Biosciences, San Jose, CA, USA) was used, and flow cytometry was performed according to a provider's protocol. Excitation was caused by a 488-nm argon laser, and emission was detected in three channels. To correct a variation in laser intensity, light scatter and mean fluorescence intensity (MFI) target values were determined using seven-color setup calibration beads (BD FACS™ 7-color setup beads; Becton Dickinson Biosciences, San Jose, Calif., USA) measurable by a forward scatter (FSC), a side scatter (SSC), and fluorescence peaks according to the provider's advice before each series is obtained. A CD45 threshold for a living cell was controlled to exclude blood platelets and debris, thereby collecting data. List mode data of 20,000 cells were collected by predetermined CD45 threshold settings. Data analysis was performed by the program FACSDiva (Becton Dickinson Biosciences, San Jose, Calif.). Flow cytometry results were collected and first input to a dedicated database system, and standard deviation (SD), a paired t-test value, and a Pearson correlation coefficient were calculated using MedCalc software. A frequency of a given population was calculated as a percentage with respect to total lymphocytes. An error was considered at a significance level of P<0.05. A link strength between general multicolor flow cytometry (FCM) and single-color multi-target FCM of lymphocytes was estimated by a paired t-test. An accuracy of each method was estimated by a coefficient of variation (CV).

### Example 1

### Single-Color 3-Target Flow Cytometry

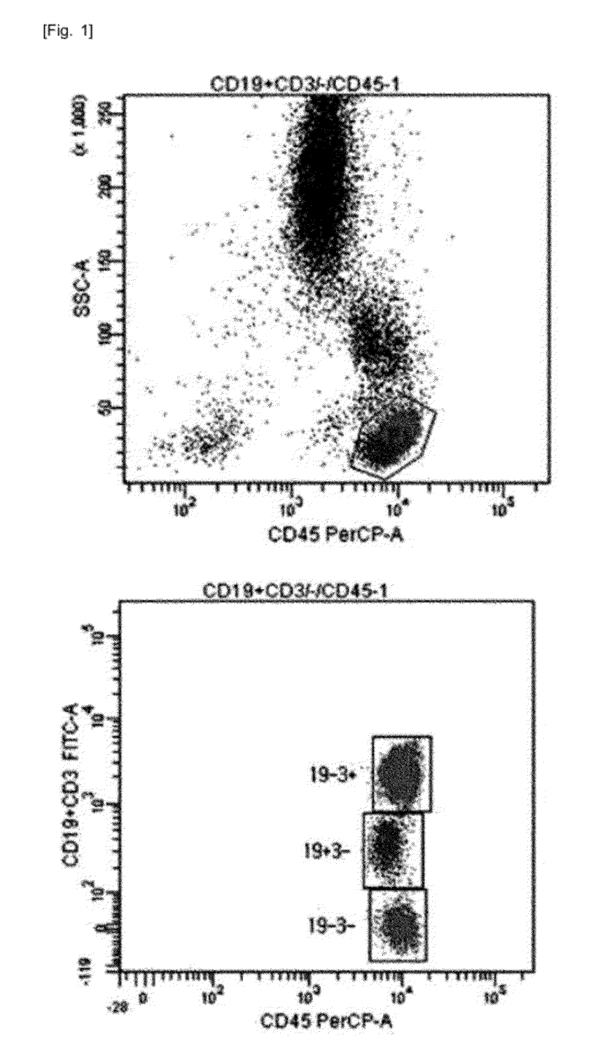

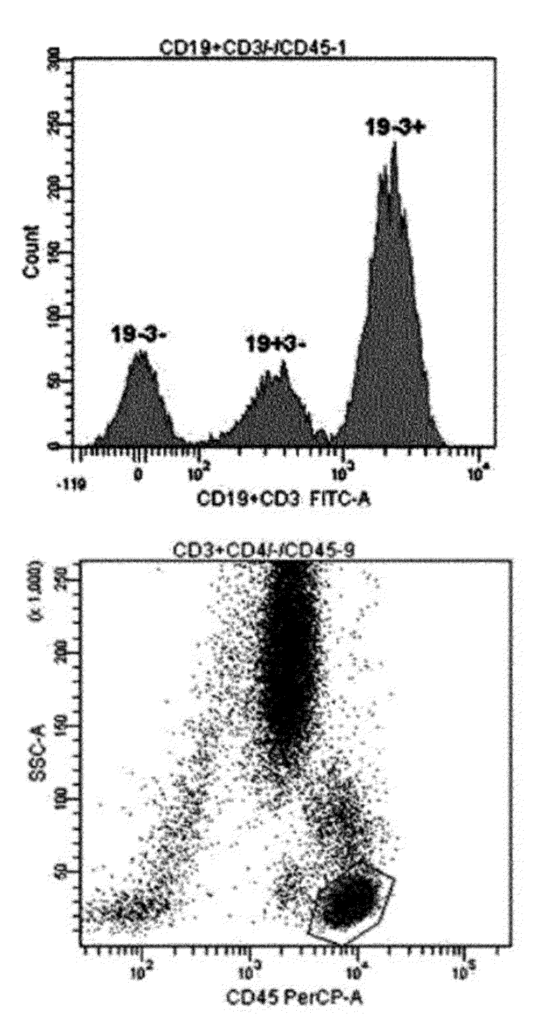

For single-color 3-target flow cytometry, two kinds of monoclonal antibodies labeled with the same fluorochrome having different intensities were used. One monoclonal antibody cocktail consisting of 0.1 μl CD3-FITC, 5 μl CD4-FITC, and 5 μl CD45-PerCP, and another monoclonal antibody cocktail consisting of 0.5 μl CD19-FITC, 5 μl CD3-FITC, and 5 μl CD45-PerCP were used (see Table 1). After incubation at room temperature for 20 minutes, erythrocytes were lysed in BD FACS™ lysing solution (Becton Dickinson Biosciences, Ontario, Canada) and incubated in a dark room for 10 minutes or more. The resultant cells were washed with phosphate buffered saline (PBS) (Medical & Biological Laboratories co., LTD, Nagoya, Japan) and then re-suspended in 0.2 mL PBS. Lymphocytes were gated using CD45/SSC (see FIG. 1). For precision analysis, two peripheral blood samples obtained from a healthy adult were analyzed 10 times each, and CVs were calculated (see Table 2). Also, for accuracy analysis, cell population data was compared with general multicolor flow cytometry results of 20 samples (see Table 3). In the general multicolor flow cytometry, a monoclonal antibody cocktail in which all antibodies have different fluorochromes and consisting of 5 μl CD5-FITC, 5 μl CD4-PE, 5 μl CD45-PerCP, 5 μl CD3-FITC, 5 μl CD19-PE, and 5 μl CD45-PerCP was used, and the flow cytometry was performed in the same way as mentioned above except for using the monoclonal antibody cocktail.

FIG. 1 shows representative dot plots and histograms of the single-color 3-target flow cytometry. The upper part of FIG. 1 shows results obtained using CD19-FITC (low intensity), CD3-FITC (high intensity), and CD45-PerCP, and the lower part of FIG. 1 shows results obtained using CD3-FITC (low intensity), CD4-FITC (high intensity), and CD45-PerCP.

As can be seen from FIG. 1, three cell populations were clearly classified by single-color 3-target flow cytometry. The method showed excellent repeatability. As can be seen from Table 2 below, CVs of lymphocyte subpopulations were 0.83 to 5.04, which were similar to general FCM data (0.74 to 4.80).

TABLE 2
Accuracy of Single-Color Multi-Target Flow Cytometry and
General Multicolor Flow Cytometry (as analyzed 10 times)

| | | |
|---|---|---|
| Cell | SM-FCM | CM-FCM |

| Population | Mean ± SD | CV | Mean ± SD | CV |
|---|---|---|---|---|
| Sample 1 | | | | |
| CD3+CD4− | 23.11 ± 0.92 | 3.98 | 24.83 ± 0.92 | 3.69 |
| CD3+CD4+ | 42.68 ± 0.87 | 2.04 | 40.45 ± 0.13 | 2.79 |
| CD19+CD3− | 19.47 ± 0.98 | 5.04 | 19.15 ± 0.69 | 3.59 |
| CD19−CD3+ | 64.81 ± 0.54 | 0.83 | 63.94 ± 0.01 | 1.57 |
| Sample 2 | | | | |
| CD3+CD4− | 32.37 ± 0.80 | 2.46 | 32.20 ± 0.04 | 3.24 |
| CD3+CD4+ | 43.06 ± 0.76 | 1.76 | 43.23 ± 0.00 | 2.32 |
| CD19+CD3− | 10.95 ± 0.45 | 4.07 | 10.93 ± 0.53 | 4.80 |
| CD19−CD3+ | 75.32 ± 1.00 | 1.32 | 75.33 ± 0.74 | 0.99 |

SM-FCM: single-color multi-target flow cytometry,
CM-FCM: conventional multicolor flow cytometry,
SD: standard deviation,
CV: coefficient of variation

As can be seen from Table 3, single-color 3-target flow cytometry showed similar results to general multicolor flow cytometry for all cell populations except for CD3+CD4-cells (P<0.05). A result of a CD3+CD4-cell population obtained using single-color 3-target flow cytometry was 28.42±8.35%, and a result of the CD3+CD4-cell population obtained using general multicolor FCM was 29.24±8.45%. A mean difference was −0.82±1.47%, which was statistically significant. However, the difference was small, and more specifically was less than a 1SD value of general FCM (0.92%).

TABLE 3
Comparison of Lymphocyte Subpopulations between Single-Color
Multi-Target Flow Cytometry and General Multicolor Flow
Cytometry (n = 20)

| | SM_FCM* | CM_FCM* | Difference | |
|---|---|---|---|---|
| Cell Population | (mean ± SD) | (mean ± SD) | (mean ± SD) | P value |
| CD3+CD4− | 28.42 ± 8.35 | 29.24 ± 8.45 | 0.82 ± 1.47 | 0.02 |
| CD3+CD4+ | 37.37 ± 10.66 | 36.72 ± 10.04 | 0.65 ± 1.58 | 0.08 |
| Total CD3+ | 65.79 ± 8.81 | 65.96 ± 8.25 | 0.17 ± 1.25 | 0.55 |
| CD19+CD3− | 13.42 ± 4.14 | 13.08 ± 3.82 | 0.34 ± 1.47 | 0.31 |
| CD19+CD3+ | 65.57 ± 8.50 | 65.96 ± 8.19 | 0.39 ± 1.54 | 0.28 |

SM-FCM* CM-FCM* Difference*;
SM-FCM: single-color multi-target flow cytometry,
CM-FCM: conventional multicolor flow cytometry

### Example 2

### Single-Color 4-Target Flow Cytometry

For single-color 4-target flow cytometry, three kinds of monoclonal antibodies labeled with the same fluorochrome having different intensities were used.

The flow cytometry was performed in the same way as in Example 1 except that a monoclonal antibody cocktail consisting of 0.1 μl CD56-PE, 0.5 μl CD19-PE, 20 μl CD4-PE and 5 μl CD45-PerCP was used.

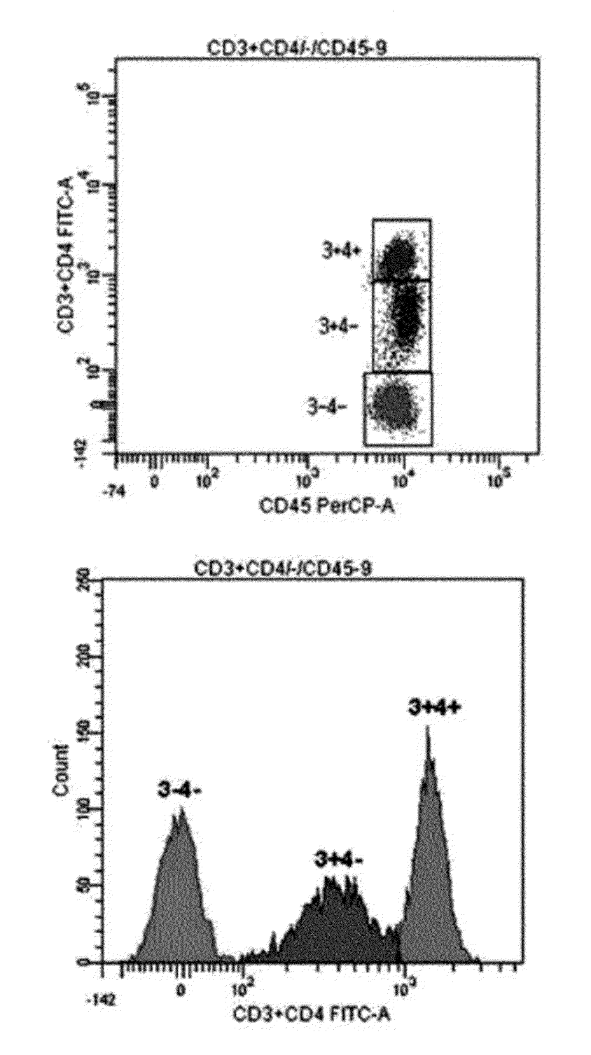

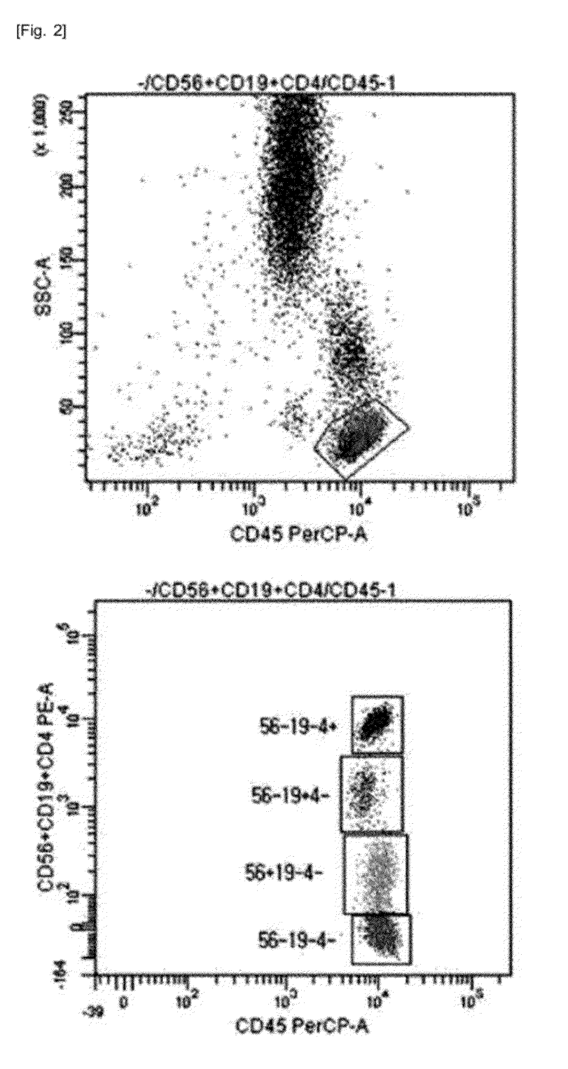

As can be seen from FIG. 2, although three kinds of monoclonal antibodies labeled with the same fluorochrome having different intensities were used, the four cell populations were clearly classified, and the profiles of four peaks in the histogram were clear enough to determine a marker with ease.

### Example 3

### Two-Color 9-Target Flow Cytometry

For 2-color 9-target flow cytometry, two kinds of monoclonal antibodies labeled with FITC having different intensities and two kinds of monoclonal antibodies labeled with PE having different intensities were used. A monoclonal antibody cocktail consisting of 0.1 μl CD5-FITC, 5 μl CD3-FITC, 0.5 μl CD19-PE, 5 μl CD4-PE, and 5 μl CD45-PerCP was used for flow cytometry of a bone marrow sample obtained from a mantle cell lymphoma patient (see Table 1). Also, to show that fluorescence intensities of antibodies used in a method according to an exemplary embodiment of the present invention can be adjusted by adjusting intensities of a fluorochrome conjugated to the respective antibodies rather than concentrations of the antibodies, a monoclonal antibody cocktail consisting of 5 μl old CD3-FITC whose fluorescence had faded (period of validity Jul. 1, 2004, 5 years 3 months prior), 5 μl CD4-FITC, 5 μl old CD25-PE (period of validity Jun. 30, 2004, 5 years 3 months prior), and 5 μl CD19-PE was used for flow cytometry of a normal peripheral blood sample (see Table 1). Except for this, the flow cytometry method was performed in the same way as in Example 1.

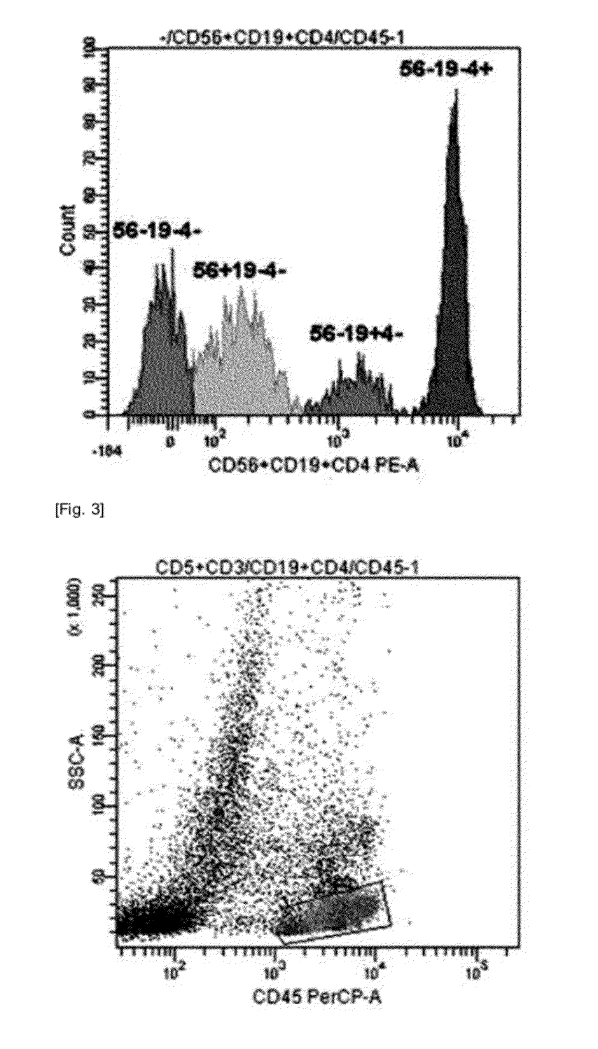

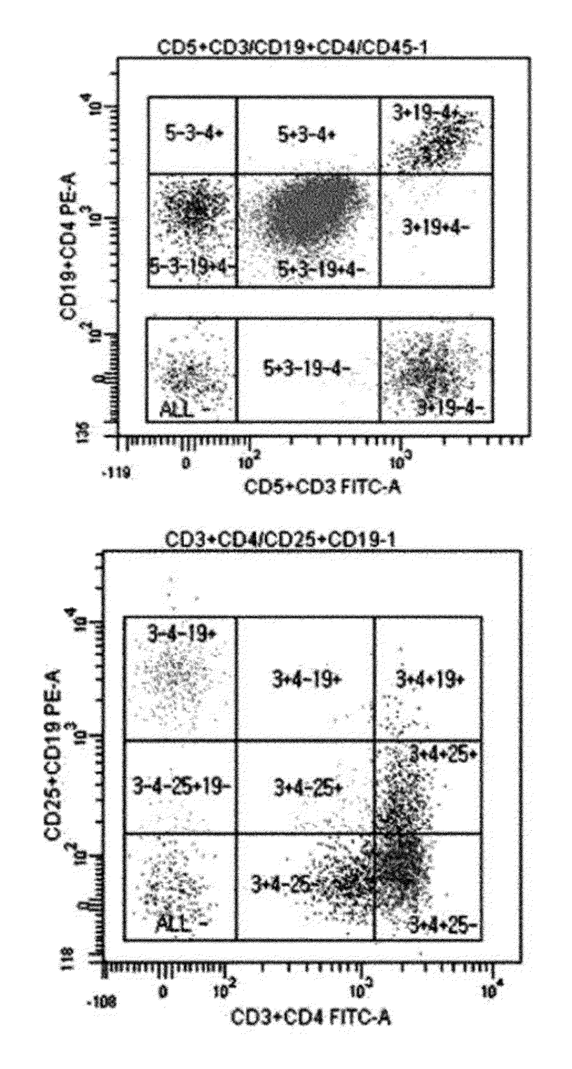

FIG. 3 shows representative dot plots of 2-color 9-target flow cytometry. As can be seen from FIG. 3, nine cell populations were clearly classified by two types of monoclonal antibodies labeled with FITC having different intensities and two types of monoclonal antibodies labeled with PE having different intensities. FIGS. 3A and 3B show results obtained using a bone marrow biopsy material of a mantle cell lymphoma patient. Lymphoma cells showing a CD5+CD19+ phenotype were clearly classified (see orange dots in FIG. 3B). FIG. 3C shows results obtained using peripheral blood of a normal individual. Although expired antibodies having reduced fluorescence were used, the lymphoma cells were classified into nine lymphocyte subpopulations.

Unlike a conventional flow cytometry method capable of classifying a positive and negative of one target using one antibody per color, the flow cytometry method according to an exemplary embodiment of the present invention adjusts several types of antibodies conjugated with a single-color fluorochrome to respectively show different fluorescence intensities, or adjusts the amounts of antibodies conjugated with a fluorochrome differently according to types of the antibodies, thereby classifying a positive and negative of multiple targets using only one color. Accordingly, even when a current flow cytometer capable of classifying a limited number of colors is used, it is possible to classify a variety of cell populations to be clinically examined.

While the invention has been shown and described with reference to certain exemplary embodiments thereof, it will be understood by those skilled in the art that various changes in form and details may be made therein without departing from the scope of the invention as defined by the appended claims.

What is claimed is:

1. A flow cytometry method, comprising:
adjusting cell populations targeted by antibodies conjugated with a same-color fluorochrome to show different fluorescence intensities according to types of the antibodies.

2. The flow cytometry method according to claim 1, wherein adjusting the cell populations to show different fluorescence intensities includes adjusting the several types of antibodies conjugated with the single-color fluorochrome to respectively show different fluorescence intensities or adjusting amounts of the antibodies conjugated with the fluorochrome differently according to the types of the antibodies, thereby adjusting fluorescence intensities of the cell populations targeted by the antibodies in different levels.

3. The flow cytometry method according to claim 2, further comprising:
incubating an antibody composition in which the several types of antibodies conjugated with the single-color fluorochrome are adjusted to respectively show different fluorescence intensities or an antibody composition in which the amounts of the antibodies conjugated with the fluorochrome are adjusted in different levels according to the types of the antibodies together with a specimen; and
gating the incubated specimen on a flow cytometer.

4. The flow cytometry method according to claim 2, wherein adjusting the several types of antibodies conjugated with the single-color fluorochrome to respectively show different fluorescence intensities, or adjusting the amounts of the antibodies conjugated with the fluorochrome differently according to the types of the antibodies is performed using single or multiple colors of fluorochromes.

5. The flow cytometry method according to claim 2, wherein adjusting the several types of antibodies conjugated with the single-color fluorochrome to respectively show different fluorescence intensities is performed by forming the single-color fluorochrome conjugated to the antibodies to have different fluorescence intensities.

6. The flow cytometry method according to claim 1, wherein the different fluorescence intensities are adjusted so that the fluorescence intensities of the different types of cell populations can differ from each other by 2 to 50 times.

7. The flow cytometry method according to claim 1, wherein the fluorochrome is selected from the group consisting of fluorescein isothiocyanate (FITC), Alexa Fluor 488, green fluorescent protein (GFP), carboxyfluorescein succinimidyl ester (CFSE), carboxyfluorescein diacetate succinimidyl ester (CFDA-SE), DyLight 488, phycoerythrin (PE), propidium iodide (PI), peridinin chlorophyll protein complex (PerCP), PerCP-Cy5.5, PE-Alexa Fluor 700, PE-Cy5 (TRI-COLOR), PE-Cy5.5, PE-Alexa Fluor 750, PE-Cy7, allophycocyanin (APC), APC-Cy7, APC-eFluor 780, Alexa Fluor 700, Cy5, Draq-5, Pacific Orange, Amine Aqua, Pacific Blue, 4′,6-diamidino-2-phenylindole HCl (DAPI), Alexa Fluor 405, eFluor 450, eFluor 605 Nanocrystals, eFluor 625 Nanocrystals, and eFluor 650 Nanocrystals.

8. The flow cytometry method according to claim 1, wherein the antibodies are directed against an antigen selected from the group consisting of CD3, CD4, CD5, CD8, CD19, CD45, and CD56.

9. The flow cytometry method according to claim 1, wherein flow cytometry is performed by a flow cytometer equipped with a detector capable of classifying three to eight colors.

10. A method of producing an antibody for flow cytometry, comprising:
conjugating respective single-color fluorochromes showing different fluorescence intensities with different types of antibodies, respectively.

11. An antibody composition, comprising:
an antibody conjugated with a fluorochrome; and
a non-conjugated antibody,
wherein an amount of the antibody conjugated with the fluorochrome is adjusted so that fluorescence intensities of cell populations can be adjusted in different levels in flow cytometry.

12. A computer-readable recording medium storing a program for analyzing distribution of cells targeted by different antibodies in a specimen, the program executing in a computer system the steps of:
recognizing different fluorescence intensities of cells targeted by different antibodies; and
classifying the cells according to the different fluorescence intensities to analyze distribution of the cells targeted by the different antibodies in a specimen.

13. A flow cytometer comprising the computer-readable recording medium of claim 12.
